(19) United States
(12) Patent Application Publication
(10) Pub. No.: US 2024/0240141 A1
(43) Pub. Date: Jul. 18, 2024

(54) RAPID AND EFFICIENT CLINICAL GRADE PIGMENT EPITHELIUM INDUCTION METHOD, KIT, AND APPLICATION

(71) Applicant: ALLIFE MEDICINE (ZHUHAI) LIMITED, Hengqin New District Zhuhai (CN)

(72) Inventors: Lida WU, Hengqin New District Zhuhai (CN); Yuchun Gu, Hengqin New District Zhuhai (CN)

(73) Assignee: ALLIFE MEDICINE (ZHUHAI) LIMITED, Hengqin New District Zhuhai (CN)

(21) Appl. No.: 18/433,083

(22) Filed: Feb. 5, 2024

Related U.S. Application Data
(63) Continuation of application No. PCT/CN2022/107890, filed on Jul. 26, 2022.

(30) Foreign Application Priority Data
Aug. 5, 2021 (CN) 202110894939.7

(51) Int. Cl. C12N 5/079; A61K 35/30

(52) U.S. Cl. CPC C12N 5/0621; A61K 35/30; C12N 2500/32; C12N 2500/38; C12N 2501/15; C12N 2501/155; C12N 2501/165; C12N 2501/415; C12N 2501/727; C12N 2506/08

(57) ABSTRACT

A rapid and efficient clinical grade pigment epithelium induction method, a kit, and an application. Provided is a method for rapidly and efficiently inducing retinal pigment epithelium (RPE). IPSCs are directionally induced in three stages, such that an RPE generation duration can be greatly shortened. Specifically, the method comprises using a culture medium containing a small molecule compound for cell culture, the small molecule compound comprising a BMP signaling pathway inhibitor, a Wnt pathway inhibitor, inhibitors for TGF-BI receptors ALK5, ALK4 and ALK7, a ROCK pathway inhibitor, a WNT signaling pathway activator, a VEGFR kinase inhibitor, a GSK signaling pathway inhibitor, a VEGFR kinase inhibitor, vitamins, and the like.

### FIELD

The present disclosure relates to the field of biotechnology, and specifically to a rapid and efficient clinical grade pigment epithelial cell induction method, kit and application.

### BACKGROUND

The eye is a vital organ for humans, while retinal degenerative diseases can cause blindness and bring great pain to patients. Retinal degenerative diseases mainly include retinitis pigmentosa (RP), macular degeneration, and hereditary retinal degeneration. These diseases have different symptoms and related pathogenic or susceptibility genes. Although these diseases vary in the etiology and pathological processes, they all involve a progressive reduction in the number of retinal cells, mainly the irreversible loss of retinal pigment epithelium (RPE) cells and photoreceptor cells, ultimately leading to the loss of visual function. RP is a hereditary disease, and more than 200 gene mutation sites related to RP have been discovered. Macular degeneration is caused by a combination of genetic changes and environmental factors. It is divided into juvenile macular degeneration and age-related macular degeneration (AMD) based on the age of onset. Clinically, RP and AMD are relatively common. RP has an incidence rate as high as 1/3000, while AMD affects more than 1/10 people over 60 years old. Currently, there are very limited drugs and methods available for retinal degenerative diseases clinically. Most of them are anti-inflammatory treatments and nerve cell trophic and protective drugs for treating retinal degenerative disease that aim to delay the progression of the disease, or drugs that inhibit blood vessel growth to treat wet AMD, etc.

However, drug treatment cannot restore damaged optic nerve cells and functional RPE cells. Cell transplantation can be used to transplant cells with specific functions and integrate them into the retina to restore their damaged functions with a broader application prospects. Currently, cell transplantation is one of the important methods for treating degenerative eye diseases. Regenerative medicine in the form of cell replacement therapies for retinal degenerative diseases holds great promise because the same therapeutic agents can be used regardless of the underlying genetic or acquired cause. Modern stem cell technology has yielded clinical-grade cell therapies, and human embryonic stem cells (hESCs) and induced pluripotent stem cells (iPSCs) are currently being studied to treat retinal degeneration. The main shortcomings of current differentiation methods are the long time required to induce differentiation (90-140 days), and the poor functionality and low survival rate of RPE cell after transplantation.

### SUMMARY

The present disclosure provides a method for quickly and efficiently inducing retinal pigment epithelial cells. Through the three stages including differentiation of ectoderm, pigment differentiation of epithelial precursor cells, and maturation of retinal pigment epithelial cells, iPSCs are directionally induced, which greatly shortens the production time of RPE cells. Each stage targets specific signaling pathways to promote iPSC differentiation.

The method provided by the present disclosure can shorten the induction differentiation time and obtain RPE cells with relatively stable functions and vitality, which can ultimately be used for transplantation treatment. Moreover, the present disclosure provides a culture medium, culture medium combination, kit and application for inducing retinal pigment epithelial cells.

In a first aspect, the present disclosure provides a method for quickly and efficiently inducing retinal pigment epithelial cells, comprising steps of inducing differentiation of ectoderm and inducing differentiation of pigment epithelial precursor cells.

Preferably, the method further comprises a step of inducing maturation of retinal pigment epithelial cells.

Preferably, the method further comprises a step of subculturing retinal pigment epithelial cells.

In the present disclosure, “pigment epithelial cells”, “retinal pigment epithelial cells” and “RPE” all have the same meaning and can be used interchangeably.

In one embodiment, the step of inducing differentiation of ectoderm comprises culturing stem cells using RDM1 culture medium and/or RDM2 culture medium.

Preferably, the stem cells include totipotent stem cells, multipotent stem cells, and unipotent stem cells.

Preferably, the stem cells are iPSCs (induced pluripotent stem cells).

Preferably, the iPSCs may be commercialized cell lines, or may be induced from donor cells, including one or more of villus cells, skin (fibroblasts and keratinocytes), amniotic fluid, extraembryonic tissue (placenta and umbilical cord), umbilical cord blood, periosteum, dental tissue, adipose tissue, neural stem cells, liver cells, mesenchymal stem cells, peripheral blood cells, mammary epithelial cells, adipose stem cells, umbilical cord matrix and placenta.

Preferably, the donor may be human or non-human.

Preferably, the non-human includes mammals (such as mice/rats, monkeys, cattle, sheep/goat, pigs, horses, chickens).

Preferably, the stem cells are human iPSCs.

Preferably, the basal culture medium of the RDM1 (“RDM1” and “RDM1 culture medium” in the present disclosure can be used interchangeably and have the same meaning) is RDM basal culture medium, and the RDM1 culture medium, in addition to the RDM basal culture medium, also comprises other substances.

Preferably, the basal culture medium of the RDM2 (“RDM2” and “RDM2 culture medium” in the present disclosure can be used interchangeably and have the same meaning) is RDM basal culture medium, and the RDM2 culture medium, in addition to the RDM basal culture medium, also comprises other substances.

Preferably, the RDM basal culture medium comprises at least one of DMEM/F12, KSR (KnockOut Serum Replacement, a serum analog), Monothioglycerol Solution (a serum-free medium for human pluripotent stem cells), Chemically Defined Lipid Concentrate, and glutamine.

More preferably, the RDM basal culture medium comprises 88% DMEM/F12, 10% KSR, 5 mM Monothioglycerol Solution, 1% Chemically Defined Lipid Concentrate, and 1% L-glutamine.

Preferably, DMEM/F12 can also be replaced by a cell culture medium selected from the group consisting of William's E medium, Neurobasal Medium, MEM medium, DMEM medium, 1640 RPMI medium, F12 medium, and a mixture thereof.

Preferably, KSR can also be replaced by a serum analog.

Preferably, the serum analog includes, but is not limited to, FBS (fetal bovine serum), horse serum, HAS (human serum albumin), or BSA (bovine serum albumin).

Preferably, the glutamine can be replaced by a glutamine substitute.

Preferably, the glutamine substitute includes GlutaMAX™ Supplement.

As used in the present disclosure, “DMEM/F-12” or “DMEM/F12” has the same meaning, which is a 1:1 mixture of DMEM and Ham's F-12. This formula combines the high concentration of glucose, amino acids and vitamins in DMEM with many components of F-12.

Preferably, DMEM/F12 includes DMEM/F-12 modified medium made by adjusting the components according to actual applications.

Preferably, the DMEM/F-12 modified medium includes, but is not limited to, DMEM-low glucose-pyruvate-glutamine free-phenol red free, DMEM/F-12-GlutaMAX™, DMEM/F-12-HEPES (DMEM/F-12 with HEPES), DMEM-low glucose-pyruvate-HEPES.

Preferably, DMEM/F-12 is DMEM/F-12 medium with HEPES, which comprises L-glutamine, HEPES, and phenol red.

Preferably, the RDM1 culture medium, in addition to the RDM basal culture medium, also comprises at least one of a BMP signaling pathway inhibitor, a Wnt pathway inhibitor, an inhibitor of TGF-β type I receptors ALK5, ALK4 and ALK7, and a ROCK pathway inhibitor.

Preferably, the BMP signaling pathway inhibitor includes noggin, Dorsomorphin, DMH1, or LDN-193189.

Preferably, the BMP signaling pathway inhibitor includes 50-200 ng/ml noggin, 2-8 μM Dorsomorphin, 10-100 μM DMH1, or 5 nM-5 μM LDN-193189.

Preferably, the BMP signaling pathway inhibitor includes 50-200 ng/ml noggin, 2-8 μM Dorsomorphin, 10-100 μM DMH1, or 5 nM-5 μM LDN-193189.

Preferably, the BMP signaling pathway inhibitor includes 50 ng/ml noggin, 2 μM Dorsomorphin, or 3 μM LDN-193189.

Preferably, the BMP signaling pathway inhibitor is 50-200 ng/mL noggin.

Preferably, the BMP signaling pathway inhibitor is 50 ng/ml noggin.

Preferably, the Wnt pathway inhibitor includes XAV-939, iCRT-3, iCRT-5, iCRT-14, IWP-4, IWR-1, or wnt-C59.

Preferably, the Wnt pathway inhibitor is 2-20 μM XAV-939.

Preferably, the Wnt pathway inhibitor is 1 μM XAV-939.

Preferably, the inhibitor of TGF-β type I receptors ALK5, ALK4 and ALK7 includes LY2109761, A83-01, SB-525334, SD-208, EW-7197, Disitertide, LY3200882, SM16, or

SB431542.

Preferably, the inhibitor of TGF-β type I receptors ALK5, ALK4 and ALK7 is 2-20 μM LY2109761.

Preferably, the inhibitor of TGF-β type I receptors ALK5, ALK4 and ALK7 is 5 μM LY2109761.

Preferably, the ROCK pathway inhibitor includes Thiazovivin or Y-27632.

Preferably, the ROCK pathway inhibitor is 0.5-20 μM Thiazovivin.

Preferably, the ROCK pathway inhibitor is 10 μM Thiazovivin;

Preferably, the RDM2 culture medium, in addition to the RDM basal culture medium, also includes at least one of a WNT signaling pathway activator, a VEGFR kinase inhibitor, and a ROCK pathway inhibitor.

Preferably, the WNT signaling pathway activator includes 6-bromoindirubin-3′-oxime (BIO).

Preferably, the WNT signaling pathway activator is 1-20 μM 6-bromoindirubin-3′-oxime (BIO).

Preferably, the WNT signaling pathway activator is 10 μM 6-bromoindirubin-3′-oxime.

Preferably, the VEGFR kinase inhibitor includes SU5402, AV-951, SU5205, or SU5408.

Preferably, the VEGFR kinase inhibitor is 1-20 μM SU5402.

Preferably, the VEGFR kinase inhibitor is 2 μM SU5402.

Preferably, the ROCK pathway inhibitor includes Thiazovivin or Y-27632.

Preferably, the ROCK pathway inhibitor is 0.5-20 μM Thiazovivin.

Preferably, the ROCK pathway inhibitor is 10 μM Thiazovivin.

In one embodiment, the step of inducing differentiation of pigment epithelial precursor cells comprises culturing RPE progenitor cells, i.e., ectodermal differentiated cells, using RDM3 and/or RDM4 culture medium;

Preferably, the RPE progenitor cells are cells cultured by the aforementioned method of inducing differentiation of ectoderm.

The “pigment epithelial precursor cells” used in the present disclosure are also the precursor cells of the aforementioned “pigment epithelial cells”, “retinal pigment epithelial cells” or “RPE”.

Preferably, the basal culture medium of the RDM3 (“RDM3” and “RDM3 culture medium” in the present disclosure can be used interchangeably and have the same meaning) is RDM basal culture medium. The RDM3 culture medium, in addition to the RDM basal culture medium, also comprises other substances.

Preferably, the RDM3 culture medium, in addition to the RDM basal culture medium, also comprises at least one of a GSK signaling pathway inhibitor, a VEGFR kinase inhibitor, a ROCK pathway inhibitor, and vitamin or a vitamin analog.

Preferably, the GSK signaling pathway inhibitor includes 6-bromoindirubin-3′-oxime (BIO).

Preferably, the GSK signaling pathway inhibitor is 1-20 μM 6-bromoindirubin-3′-oxime (BIO).

Preferably, the GSK signaling pathway inhibitor is 10 μM 6-bromoindirubin-3′-oxime.

Preferably, the VEGFR kinase inhibitor includes SU5402, AV-951, SU5205, or SU5408.

Preferably, the VEGFR kinase inhibitor is 1-20 μM SU5402.

Preferably, the VEGFR kinase inhibitor is 2 μM SU5402.

Preferably, the ROCK pathway inhibitor includes Thiazovivin or Y-27632.

Preferably, the ROCK pathway inhibitor is 0.5-20 μM Thiazovivin.

Preferably, the ROCK pathway inhibitor is 10 μM Thiazovivin.

Preferably, the vitamin or the vitamin analog includes biotin, choline chloride, D-calcium pantothenate, folic acid, inositol, nicotinamide, pyridoxine hydrochloride, riboflavin, coenzyme Q10, putrescine dihydrochloride, Vitamin A, Vitamin B, Vitamin C, Vitamin D, Vitamin E, Vitamin K, Vitamin H, Vitamin P, Vitamin M, Vitamin T, Vitamin U, or water-soluble vitamins.

Preferably, the vitamin or the vitamin analog includes Vitamin B.

More specifically, the vitamin or the vitamin analog is Vitamin B3.

Preferably, the vitamin or the vitamin analog is 1-20 mM Vitamin B3.

Preferably, the vitamin or the vitamin analog is 10 mM Vitamin B3.

Preferably, the RDM4 culture medium comprises DMEM/F12, KSR, N2 medium, glutamine and vitamin.

Preferably, the DMEM/F12 or N2 medium can also be replaced by a cell culture medium selected from the group consisting of William's E medium, Neurobasal Medium, MEM medium, DMEM medium, 1640 RPMI medium, F12 medium, and a mixture thereof.

Preferably, KSR can also be replaced by a serum analog including but not limited to FBS (fetal bovine serum), horse serum, HAS (human serum albumin), or BSA (bovine serum albumin).

Preferably, the glutamine can be replaced by a glutamine substitute.

Preferably, the glutamine substitute includes GlutaMAX™ Supplement.

More preferably, the RDM4 culture medium comprises 89% DMEM/F12, 10% KSR, 1% N2 medium, 1% L-glutamine and 10 mM Vitamin B3.

Preferably, the step of inducing maturation of retinal pigment epithelial cells comprises culturing pigment epithelial precursor cells using RMM culture medium.

Preferably, the RMM culture medium comprises DMEM/F12, B27 medium, and glutamine.

Preferably, the RMM culture medium comprises 97% DMEM/F12, 2% B27 culture medium, and 1% L-glutamine.

Preferably, the DMEM/F12 or B27 medium can also be replaced by a cell culture medium selected from the group consisting of William's E medium, Neurobasal Medium, MEM medium, DMEM medium, 1640 RPMI medium, F12 medium, and a mixture thereof.

Preferably, KSR can also be replaced by a serum analog, including but not limited to FBS (fetal bovine serum), horse serum, HAS (human serum albumin), or BSA (bovine serum albumin).

Preferably, the glutamine can be replaced by a glutamine substitute.

Preferably, the glutamine substitute includes GlutaMAX™ Supplement.

Preferably, the step of subculturing retinal pigment epithelial cells comprises culturing mature retinal pigment epithelial cells using REM culture medium.

Preferably, the REM culture medium comprises DMEM/F12, KSR, glutamine, and β-mercaptoethanol.

Preferably, the REM culture medium comprises 79% DMEM/F12, 20% KSR, 1% L-glutamine, and 50 μM β-mercaptoethanol.

Preferably, DMEM/F12 can also be replaced by a cell culture medium selected from the group consisting of William's E medium, Neurobasal Medium, MEM medium, DMEM medium, 1640 RPMI medium, F12 medium, and a mixture thereof.

Preferably, KSR can also be replaced by a serum analog, including but not limited to FBS (fetal bovine serum), horse serum, HAS (human serum albumin), or BSA (bovine serum albumin).

Preferably, the glutamine can be replaced by a glutamine substitute.

Preferably, the glutamine substitute includes GlutaMAX™ Supplement.

Preferably, the β-mercaptoethanol can also be replaced by a reducing agent, including but not limited to β-mercaptoethanol, dithiothreitol, dithioerythritol, reduced glutathione, cysteine, thiocarbamate, sodium disulfonate, ascorbate, tin dichloride or sodium borohydride.

Preferably, the concentrations mentioned in the present disclosure are final concentrations, and the percentages are percentages of volume.

Preferably, the frequency of medium replacement when using any of the RDM1, RDM2, RDM3, RDM4, RMM or REM medium is adjusted according to the growth status of cells; preferably, the frequency of medium replacement is daily.

Preferably, the total number of days of using the RDM1 culture medium is 3-9 days.

Preferably, the total number of days of using the RDM1 culture medium is 6 days.

Preferably, the total number of days of using the RDM2 culture medium is 2-8 days.

Preferably, the total number of days of using the RDM2 culture medium is 5 days.

Preferably, the total number of days of using the RDM3 culture medium is 1-7 days.

Preferably, the total number of days of using the RDM3 culture medium is 4 days.

Preferably, the total number of days of using the RDM4 culture medium is 3-9 days.

Preferably, the total number of days of using the RDM4 culture medium is 6 days.

Preferably, the total number of days of using the RMM culture medium is 6-12 days.

Preferably, the total number of days of using the RMM culture medium is 9 days.

Preferably, the method further comprises a step of cell detection.

Preferably, the cell detection can be one or more of cell activity detection, immune-based detection, flow cytometry detection, colorimetric detection, gold nanoparticle-based detection, fluorescence detection, ultraviolet detection, and cell marker detection.

Preferably, the method uses methods for cell culture that are commonly used by those skilled in the art to process cells. The cell culture is any form of cell preparation, cell sorting, cell clone culture, cell expansion culture, cell enrichment, cell purification, cell engineering, three-dimensional cell culture, cell fermentation, tissue culture, and organ culture performed in vitro using a culture medium.

Preferably, the cell culture can be performed in an incubator or other environment suitable for cell growth.

Preferably, the incubator is a CO₂ incubator.

Preferably, the incubator is a constant temperature incubator; more preferably, the incubator has a constant temperature of 37° C.

In a second aspect, the present disclosure provides a method for quickly and efficiently inducing RPE progenitor cells, comprising culturing stem cells using a culture medium comprising a small molecule compound.

The small molecule compound comprises any one or more of a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, and a VEGFR kinase inhibitor.

Preferably, the culture medium comprising the small molecule compound is the aforementioned RDM1 culture medium and/or RDM2 culture medium.

In a third aspect, the present disclosure provides a method for inducing pigment epithelial precursor cells, comprising culturing cells using a culture medium comprising a small molecule compound.

The small molecule compound comprises any one or more of a GSK signaling pathway inhibitor, a VEGFR kinase inhibitor, a ROCK pathway inhibitor, and vitamin or a vitamin analog.

Preferably, the culture medium comprising the small molecule compound is the RDM3 culture medium according to claim 3.

Preferably, the cell culture is culture of RPE progenitor cells.

Preferably, the RPE progenitor cells are prepared by the aforementioned method of quickly and efficiently inducing RPE progenitor cells.

Preferably, the method further comprises culturing cells using the aforementioned RDM4 culture medium.

In a fourth aspect, the present disclosure provides a culture medium, which is selected from any one of the following: the aforementioned RDM1 culture medium, RDM2 culture medium, RDM3 culture medium, RDM4 culture medium, RMM culture medium, and REM culture medium.

In a fifth aspect, the present disclosure provides a culture medium combination, which is selected from any combination of the following: the aforementioned RDM1 culture medium, RDM2 culture medium, RDM3 culture medium, RDM4 culture medium, RMM culture medium, REM culture medium.

In a sixth aspect, the present disclosure provides a kit for inducing retinal pigment epithelial cells, wherein the kit comprises at least one of the following substances: a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, a VEGFR kinase inhibitor, a GSK signaling pathway inhibitor, a VEGFR kinase inhibitor, and vitamin or a vitamin analog.

Alternatively, the kit comprises reagents for preparing any one or more of the aforementioned RDM1 culture medium, RDM2 culture medium, RDM3 culture medium, RDM4 culture medium, RMM culture medium, and REM culture medium.

Preferably, the RDM1 culture medium, RDM2 culture medium, RDM3 culture medium, RDM4 culture medium, RMM culture medium and REM culture medium of the present disclosure can be a self-prepared medium or a commercial medium.

Preferably, the kit also comprises an instrument required for culturing cells.

Preferably, the instrument includes, but is not limited to, culture vessels (such as culture plates, petri dishes, culture bottles), incubators (including CO₂ incubators), biological safety cabinets, centrifuges, water bath instruments, refrigerators, pure water equipment, microscopes, drying ovens, cell freezing storage, or sterilizers.

In a seventh aspect, the present disclosure provides application of the aforementioned RDM1 culture medium, RDM2 culture medium, RDM3 culture medium, RDM4 culture medium, RMM culture medium, REM culture medium, culture medium combination, kit, a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, a VEGFR kinase inhibitor, a GSK signaling pathway inhibitor, or vitamin or a vitamin analog in inducing retinal pigment epithelial cells.

In an eighth aspect, the present disclosure provides retinal pigment epithelial cells prepared by the aforementioned method and their use in the manufacture of a medicament for treating an ophthalmic disease.

In a ninth aspect, the present disclosure provides a method for treating an ophthalmic disease, comprising preparing retinal pigment epithelial cells using the aforementioned method.

Preferably, the method for treating an ophthalmic disease further comprises cell transplantation.

Preferably, the ophthalmic disease includes retinal degenerative disease; and the main symptoms of the retinal degenerative disease include irreversible loss of retinal pigment epithelial cells, ultimately leading to loss of visual function.

Preferably, the retinal degenerative disease mainly includes retinitis pigmentosa (RP), macular degeneration, Leber disease (also known as Leber congenital amaurosis), Usher syndrome, or retinal atrophy (including retinal atrophy caused by lesions that damage the retina or caused by genetic factors).

Preferably, the macular degeneration includes juvenile macular degeneration (Stargarde, also known as congenital macular degeneration) and age-related macular degeneration (AMD).

In a tenth aspect, the present disclosure provides the following applications:
1) Application of any one of a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, a VEGFR kinase inhibitor, a GSK signaling pathway inhibitor, or vitamin or a vitamin analog in inducing retinal pigment epithelial cells;
2) Application of any one of a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, a VEGFR kinase inhibitor, a GSK signaling pathway inhibitor, or vitamin or a vitamin analog in inducing retinal pigment epithelial cells;
3) Application of RDM1 culture medium, RDM2 culture medium, a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, a VEGFR kinase inhibitor, or the aforementioned kit in inducing RPE progenitor cells; or
4) Application of RDM3 culture medium, RDM4 culture medium, a GSK signaling pathway inhibitor, a VEGFR kinase inhibitor, a ROCK pathway inhibitor, vitamin or a vitamin analog, or the aforementioned kit in inducing pigment epithelial precursor cells.

In an eleventh aspect, the present disclosure provides the following cell populations:
1) A cell population prepared by the aforementioned method of inducing RPE progenitor cells, wherein the proportion of cells expressing PAX6 and RPE65 in the cell population is at least 5%, preferably at least 10%;
2) A cell population prepared by the aforementioned method of inducing pigment epithelial precursor cells, wherein the proportion of cells expressing PAX6 and RPE65 in the cell population is at least 10%, preferably at least 20%;
3) A cell population prepared by the aforementioned method of inducing retinal pigment epithelial cells, wherein the proportion of positive cells expressing ZO-1, RPE65, Pax6, and CRALBP in the cell population is at least 80%; or
4) A cell population prepared by the aforementioned method of inducing retinal pigment epithelial cells, wherein the expression levels of TYRP2, PEDF, PMEL17, RPE65, and CRALBP in the cell population are increased by at least 100 times relative to iPSCs.

### BRIEF DESCRIPTION OF DRAWINGS

FIG. 1 shows the changes in cell morphology under a 4× light microscope on days 2, 7 and 12; A of FIG. 1: Day 2, B of FIG. 1: Day 7, C of FIG. 1: Day 14.

FIG. 2 shows the changes in cell morphology under a 4× light microscope on days 14, 18 and 24; A of FIG. 2: Day 14, B of FIG. 2: Day 18, C of FIG. 2: Day 24.

FIG. 3 shows the changes in cell morphology under a light microscope at 4×, 10×, and 20× on day 52; A of FIG. 3: 4×, B of FIG. 3: 10×, C of FIG. 3: 20×.

FIG. 4 shows the overall morphology of the cells under the naked eye on day 52.

FIG. 5 shows the detection of PAX6 and RPE65 double-positive cells on days 6, 12 and 24; where the first column shows DAPI staining, the second column shows PAX6 staining, the third column shows RPE65 staining, and the fourth column shows the fusion of PAX6 and RPE65.

FIG. 6 shows a statistical result of the relative expression of mRNAs of RPE progenitor cell-related genes on day 12.

FIG. 7 shows the result of immunofluorescence detection of RPE progenitor cell-related genes on day 36; where A of FIG. 7 shows the cell morphology under white light, B of FIG. 7 shows the result of ZO1 staining, C of FIG. 7 shows the result of PAX6 and RPE65 staining, and D shows the result of ZO1 and CRALBP staining.

FIG. 8 shows a statistical result of the relative expression of mRNAs of RPE cell-related genes on day 36.

FIG. 9 shows a statistical result of the relative expression of PAX6 mRNA in cells on day 6 when using different BMP inhibitors.

### DETAILED DESCRIPTION

The present disclosure will be further described below in conjunction with the examples. The following descriptions are only preferred embodiments of the present disclosure, and are not intended to limit the present disclosure in other forms. Any skilled person familiar with the art may make use of the technical content disclosed above to modify it into equivalent embodiments with equivalent changes. Any simple modifications or equivalent changes made to the following embodiments based on the technical essence of the present disclosure without departing from the content of the present disclosure fall within the protection scope of the present disclosure.

### General Method 1. Immunofluorescence Detection Steps

1. Soaking a slide with cells on it in a 24-well culture plate three times with PBS for 3 min each time;
2. Fixing the slide with 4% paraformaldehyde at room temperature for 15 min, and soaking the slide in PBS 3 times for 3 min each time;
3. Permeabilizing with 0.5% Triton X-100 (prepared with 5% BSA) at room temperature for 20 min (this step is omitted for antigens expressed on the cell membrane);
4. Using 5% BSA for blocking at room temperature for 1 hour;
5. Removing the blocking solution, adding 400 μl of primary antibody to each well, and incubating overnight at 4° C.;
6. After 12 hours, adding fluorescent secondary antibody: soaking in PBST 3 times for 5 min each time, adding diluted fluorescent secondary antibody, incubating at room temperature for 1 h, and soaking in PBST 3 times for 5 min each time;
9. Counter-staining nuclei: adding DAPI dropwise, incubating in the dark for 5 min, staining the nuclei of the specimen, and washing away excess DAPI with PBST 5 min×4 times;
10. Adding 500 μl PBS and putting on the machine to film.
   General Method 2. qPCR Detection of Gene Expression Levels

1. Collect 200W cells, adding 1 ml TRIZOL, extracting RNA and determining the RNA concentration. Taking 1 μg RNA and reverse-transcribing it into cDNA. Premixing according to the system as follows in Table 1.

TABLE 1
PCR reaction system

| Component | Volume | |
|---|---|---|
| Forward Primer (10 μm) | 0.4 | μl |
| Reverse Primer (10 μm) | 0.4 | μl |
| 2×TransStar Top/Tip Green qPCR | 10 | μl |
| SuperMix | | |
| Nuclease-free Water | 7.2 | μl |
| cDNA | 2 | μl |
| Total volμme | 20 | μl |

2. Then putting the above system into the Light cycler instrument for reaction according to the 3-step method with a number of cycles of 45. The reaction system is as follows in Table 2:

TABLE 2
PCR reaction procedure

| Temp | Time | |
|---|---|---|
| 94° C. | 30 s | 45 |
| 94° C. | 5 s | cycles |
| 55° C. | 15 s | |
| 72° C. | 10 s | |

### Example 1 RPE Differentiation Process and Detection Results

The reagents used in the present disclosure are shown in Table 3 below:

TABLE 3
Reagents used in the present disclosure

| Name | Company | Item No. |
|---|---|---|
| DMEM/F-12 with HEPES | GIBCO | 11330032 |
| matrigel | Corning | 354277 |
| KnockOut Serum Replacement (KSR) | GIBCO | A3181502 |
| L-Glutamine (100×) | GIBCO | 35050061 |
| LDN193189 | sigma | SML0559 |
| LY2109761 | abmole | M2081 |
| XAV-939 | abmole | M1796 |
| Thiazovivin | abmole | M1856 |
| 6-bromoindirubin-3'-oxime (BIO) | abmole | M7627 |
| SU 5402 | biogems | 2159233 |
| Vitamin B3 | sigma | N3376-100G |
| N2 supplement | GIBCO | 17502001 |
| B-27 ™ Supplement, XenoFree, | Life | A3353501 |
| minus vitamin A | technologies | |
| 0.25% Trypsin-EDTA(1×) | Thermo | 25200072 |
| Chemically Defined Lipids | GIBCO | 0920239SA |
| Monothioglycerol Solution | Sigma | M6145 |

### Culturing Process:

### (1) Inducing Differentiation of Ectoderm

1. On day 0, iPSCs were plated at a density of 1.0×10³-5.0×10⁵/cm². In this example, the cells were plated at a density of 5.0×10³/cm² and then cultured in a 37° C./5% CO₂ cell culture incubator with RDM1 as the culture medium.

The basal culture medium of RDM1 comprised 88% DMEM/F12, 10% KSR, 5 mM Monothioglycerol Solution, 1% Chemically Defined Lipid Concentrate, and 1% L-glutamine.

Besides, the RDM1 culture medium also comprised:
1) BMP signaling pathway inhibitor: 50 ng/ml noggin
2) Wnt pathway inhibitor: 1 μM XAV-939;
3) Inhibitor of TGF-β type I receptors ALK5, ALK4 and ALK7: 5 μM LY2109761
4) ROCK pathway inhibitor: 10 μM Thiazovivin.

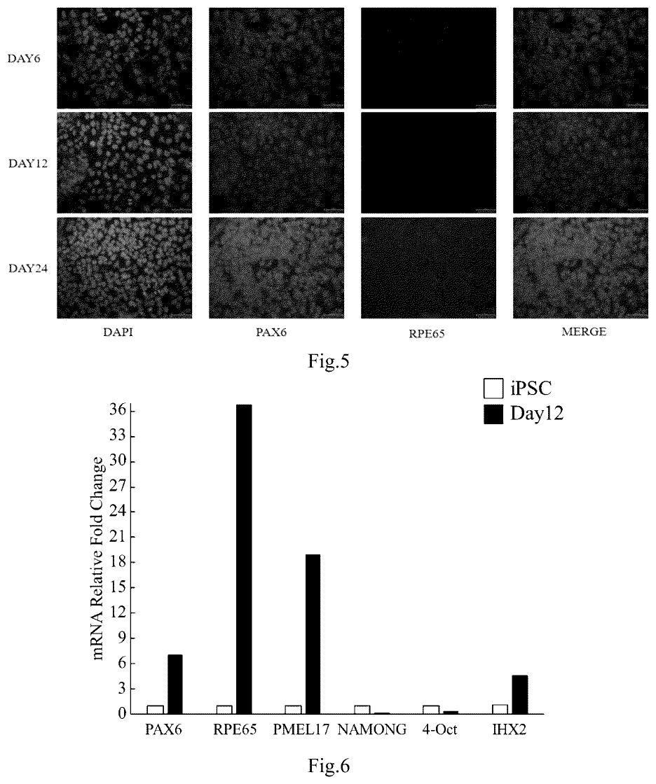

2. From day 1 to day 6, the RDM1 culture medium was renewed every day. By day 6, the successful differentiation was marked by at least 5% PAX6 and RPE65 double-positive cells. The detection method is shown in General Method 1, and the results are shown in the first row of FIG. 5.

3. From day 7 to day 12, RDM2 culture medium was used and renewed every day. The basal culture medium of RDM2 comprised 88% DMEM/F12, 10% KSR, 5 mM Monothioglycerol Solution, 1% Chemically Defined Lipid Concentrate, and 1% L-glutamine.

Besides, the RDM2 culture medium also comprised:
1) WNT signaling pathway activator: 10 μM 6-bromoindirubin-3′-oxime (BIO).
2) VEGFR kinase inhibitor: 2 μM SU5402
3) ROCK pathway inhibitor: 10 μM Thiazovivin.

The successful differentiation in this example was marked by the generation of at least 10% PAX6 and RPE65 double-positive cells. The detection method is shown in General Method 1, and the detection results are shown in the second row of FIG. 5. Alternatively, the successful differentiation in this example was marked by that the expression levels of PAX6, RPE65, IHX2, and pmel17 were increased by 5 times. The detection method is shown in General Method 2, and the results are shown in FIG. 6.

### (2) Inducing Differentiation of Pigment Epithelial Precursor Cells

4. From day 13 to day 17, RDM3 culture medium was used and renewed every day. The basal culture medium of RDM3 comprised 88% DMEM/F12, 10% KSR, 5 mM Monothioglycerol Solution, 1% Chemically Defined Lipid Concentrate, and 1% L-glutamine.

Besides, the RDM3 culture medium also comprised:
1) GSK signaling pathway inhibitor: 10 μM 6-bromoindirubin-3′-oxime (BIO);
2) VEGFR kinase inhibitor: 2 μM SU5402;
3) ROCK pathway inhibitor: 10 μM Thiazovivin;
4) Vitamin or vitamin analog: 10 mM B3.

5. From day 18 to day 24, RDM4 culture medium was used and renewed every day. The basal culture medium of RDM4 comprised 89% DMEM/F12, 10% KSR, 1% N2 medium, 1% L-glutamine and 10 mM Vitamin B3.

The successful differentiation in this example was marked by the generation of at least 20% PAX6 and RPE65 positive cells. The detection method is shown in General Method 1, and the detection results are shown in the third row of FIG. 5.

### (3) Inducing Maturation of Retinal Pigment Epithelial Cells

6. From day 25 to day 36, RMM culture medium was used and renewed every day. The basal culture medium of RMM comprised 97% DMEM/F12, 2% B27 medium, and 1% L-glutamine.

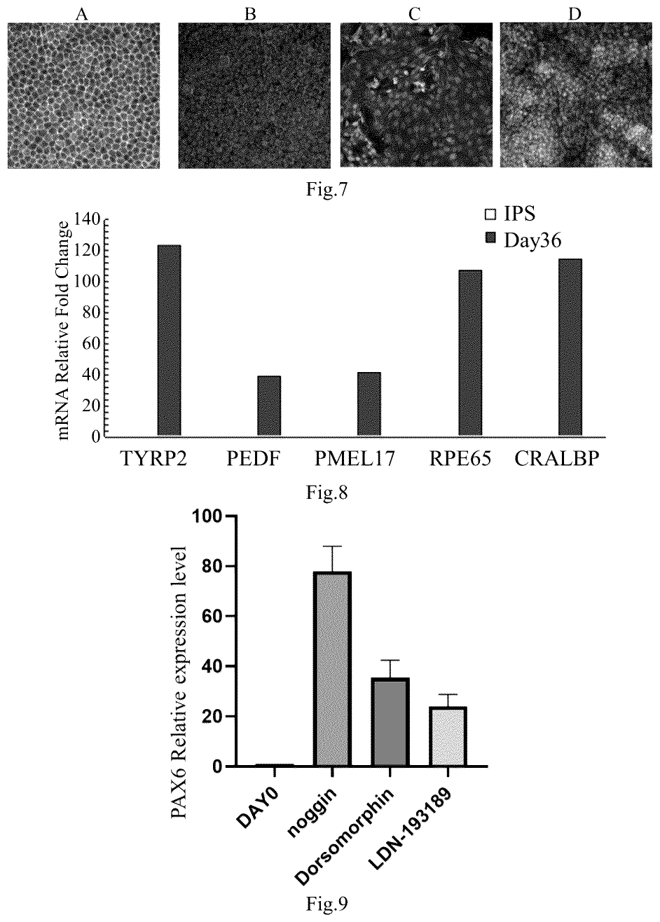

The successful differentiation in this example was marked by the generation of at least 80% ZO-1, RPE65, Pax6, and CRALBP-positive cells, and no less than 1% of cells producing black pigmentation. The detection method is shown in General Method 1, and the detection results are shown in FIG. 7. Alternatively, the successful differentiation in this example was marked by that the expression levels of TYRP2, PEDF, PMEL17, RPE65, and CRALBP were increased by at least 100 times detected by qPCR. The detection method is shown in General Method 2, and the detection results are shown in FIG. 8.

### (4) Subculturing Retinal Pigment Epithelial Cells

7. After day 37, the old culture medium was removed. The cells were washed twice with room temperature DPBS, then added with 1 mL of 0.25% Trypsin-EDTA preheated at 37° C., and placed in a 37° C./5% CO₂ cell culture incubator for 10 min. Gaps were observed between individual cells under a microscope.

8. Trypsin-EDTA was discarded, and 3 ml of REM culture medium was added to terminate digestion;

9. The culture solution was filtered with a 35 μM filter, transferred to a 15 ml centrifuge tube and centrifuged at 1000 rpm for 5 min at room temperature.

10. After the supernatant was discarded, the cells were gently pipetted and resuspended in REM culture medium, counted, and plated into a matrigel-coated six-well plate.

11. From day 38 to day 51, REM was renewed every other day until the cells were collected and frozen.

In this example, the REM culture medium comprised 79% DMEM/F12, 20% KSR, 1% L-glutamine, and 50 μM β-mercaptoethanol.

### Test Results:

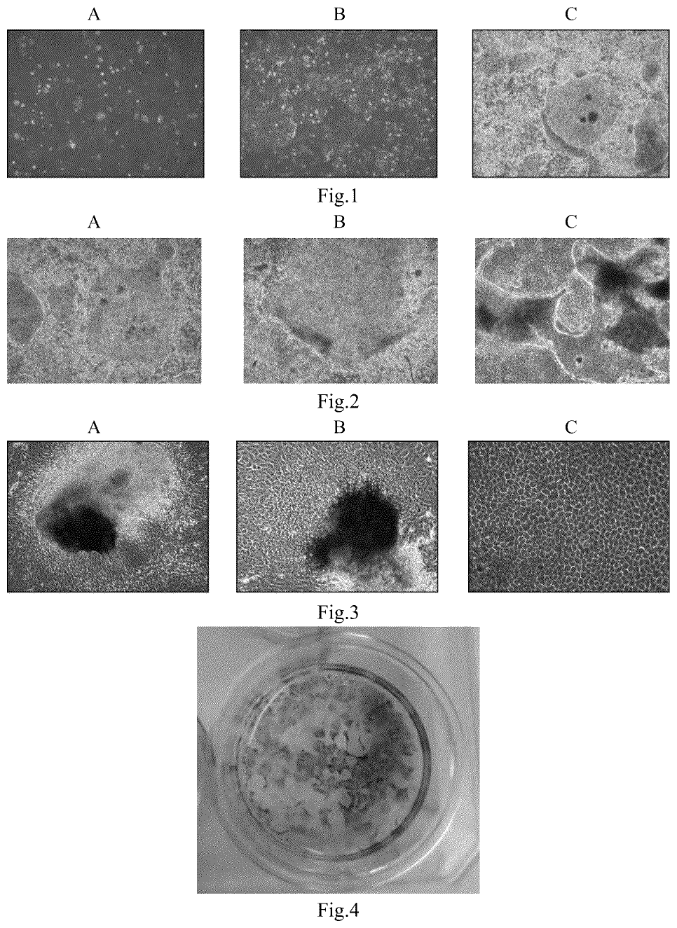

The changes in cell morphology were observed under a 4× light microscope. The cell morphology on days 2, 7 and 12 is shown in FIG. 1. On day 2, the cells showed colony growth. On day 7, the cells expanded and showed an obvious epithelioid morphology. On day 12, some epithelioid cells began to accumulate and grow. The cell morphology on days 14, 18 and 24 is shown in FIG. 2. On day 14, the epithelioid cells showed colony accumulation growth. On day 24, the epithelioid cells showed colony accumulation growth. The cell morphology changed and the cell boundaries became more distinct. On day 24, the epithelioid cells showed colony accumulation growth and the cell morphology showed irregular polygons.

The changes in cell morphology under a light microscope at 4×, 10×, and 20× on day 52 are shown in FIG. 3. Under the 4× and 10× microscopes, the epithelioid cells showed colony accumulation growth, the cell morphology changed, and the cell boundaries became more distinct. Under the 20× light microscope, the cells showed a typical regular hexagonal shape. The overall morphology of cells on day 52 is shown in FIG. 4. About 80% of the cells showed black pigmentation to the naked eye.

FIG. 5 shows the detection of PAX6 and RPE65 double-positive cells on days 6, 12 and 24. On day 6, successful differentiation produced at least 5% PAX6 and RPE65 double-positive cells; on day 12, successful differentiation produced at least 10% PAX6 and RPE65 double-positive cells; on day 24, successful differentiation produced at least 20% PAX6 and RPE65 double-positive cells.

FIG. 6 shows the detection of RPE progenitor cell-related genes on day 12. The expression levels of PAX6, RPE65, IHX2, and pmel17 were increased by at least 5 times relative to iPSCs. OCT4 and NANOG are both iPSC marker genes. Their expression in RPE progenitor cells was low.

FIG. 7 shows the detection of RPE cell-related genes on day 36. The differentiation produced at least 80% ZO-1, RPE65, Pax6, and CRALBP-positive cells

FIG. 8 shows the detection of RPE cell-related genes on day 36. The expression levels of TYRP2, PEDF, PMEL17, RPE65, and CRALBP were increased by at least 100 times relative to iPSCs.

### Example 2. Induction of Cells Using Different BMP Signaling Pathway Inhibitors in RDM1 Culture Medium

iPSCs were cultured according to the method of Example 1. 50 ng/mL noggin, 2 μM Dorsomorphin or 3 μM LDN-193189 was added to the RDM1 culture medium as three sets of parallel controls. The expression level of PAX6 in the cells on day 6 was detected.

The results are shown in FIG. 9: Day 0 was used as a control. The expression level of PAX6 was detected after 6 days of cell differentiation. Among them, the application of noggin had the best effect and the highest expression level of PAX6.

1. A culture medium, selected from the group consisting of:
(1) an RDM1 culture medium, comprising at least one of a BMP signaling pathway inhibitor, a Wnt pathway inhibitor, an inhibitor of TGF-β type I receptors ALK5, ALK4 and ALK7, and a ROCK pathway inhibitor;
preferably, the BMP signaling pathway inhibitor is selected from the group consisting of noggin, Dorsomorphin, DMH1, and LDN-193189;
preferably, the BMP signaling pathway inhibitor is selected from the group consisting of 50-200 ng/ml noggin, 2-8 μM Dorsomorphin, 10-100 μM DMH1, and 5 nM-5 μM LDN-193189;
preferably, the BMP signaling pathway inhibitor is selected from the group consisting of 50 ng/ml noggin, 2 μM Dorsomorphin, and 3 μM LDN-193189;
preferably, the BMP signaling pathway inhibitor is noggin;
preferably, noggin is at a working concentration of 50 ng/ml;
preferably, the Wnt pathway inhibitor is selected from the group consisting of XAV-939, iCRT-3, iCRT-5, iCRT-14, IWP-4, IWR-1, and wnt-C59;
preferably, the Wnt pathway inhibitor is 2-20 μM XAV-939;
preferably, the inhibitor of TGF-β type I receptors ALK5, ALK4 and ALK7 is selected from the group consisting of LY2109761, A83-01, SB-525334, SD-208, EW-7197, Disitertide, LY3200882, SM16, and SB431542;
preferably, the inhibitor of TGF-β type I receptors ALK5, ALK4 and ALK7 is 2-20 μM LY2109761;
preferably, the inhibitor of TGF-β type I receptors ALK5, ALK4 and ALK7 is 5 μM LY2109761;
preferably, the ROCK pathway inhibitor is selected from the group consisting of Thiazovivin and Y-27632;
preferably, the ROCK pathway inhibitor is 0.5-20 μM Thiazovivin;
preferably, the ROCK pathway inhibitor is 10 μM Thiazovivin;
preferably, a basal culture medium of the RDM1 culture medium is RDM basal culture medium, wherein the RDM basal culture medium comprises DMEM/F12, KSR, Monothioglycerol Solution, Chemically Defined Lipid Concentrate, and glutamine;
preferably, DMEM/F12 is replaced by a cell culture medium selected from the group consisting of William's E medium, Neurobasal Medium, MEM medium, DMEM medium, 1640 RPMI medium, F12 medium, and a mixture thereof;
preferably, KSR is replaced by a serum analog, wherein the serum analog is selected from the group consisting of FBS, horse serum, HAS, and BSA;
preferably, the glutamine is replaced by GlutaMAX™ Supplement or L-glutamine;
preferably, the RDM basal culture medium comprises 88% DMEM/F12, 10% KSR, 5 mM Monothioglycerol Solution, 1% Chemically Defined Lipid Concentrate, and 1% L-glutamine;
(2) an RDM2 culture medium, comprising at least one of a WNT signaling pathway activator, a VEGFR kinase inhibitor, and a ROCK pathway inhibitor;
preferably, the WNT signaling pathway activator is 6-bromoindirubin-3′-oxime;
preferably, the WNT signaling pathway activator is 1-20 μM 6-bromoindirubin-3′-oxime;
preferably, the WNT signaling pathway activator is 10 μM 6-bromoindirubin-3′-oxime;
preferably, the VEGFR kinase inhibitor is selected from the group consisting of SU5402, AV-951, SU5205, SU5408;
preferably, the VEGFR kinase inhibitor is 1-20 μM SU5402;
preferably, the VEGFR kinase inhibitor is 2 μM SU5402;
preferably, the ROCK pathway inhibitor is selected from the group consisting of Thiazovivin and Y-27632;
preferably, the ROCK pathway inhibitor is 0.5-20 μM Thiazovivin;
preferably, the ROCK pathway inhibitor is 10 μM Thiazovivin;
preferably, a basal culture medium of the RDM1 culture medium is RDM basal culture medium, wherein the RDM basal culture medium comprises DMEM/F12, KSR, Monothioglycerol Solution, Chemically Defined Lipid Concentrate, and glutamine;
preferably, DMEM/F12 is replaced by a cell culture medium selected from the group consisting of William's E medium, Neurobasal Medium, MEM medium, DMEM medium, 1640 RPMI medium, F12 medium, and a mixture thereof;
preferably, KSR is replaced by a serum analog, wherein the serum analog is selected from the group consisting of FBS, horse serum, HAS, and BSA;
preferably, the glutamine is replaced by GlutaMAX™ Supplement or L-glutamine;
preferably, the RDM basal culture medium comprises 88% DMEM/F12, 10% KSR, 5 mM Monothioglycerol Solution, 1% Chemically Defined Lipid Concentrate, and 1% L-glutamine; and
(3) an RDM3 culture medium, comprising at least one of a GSK signaling pathway inhibitor, a VEGFR kinase inhibitor, a ROCK pathway inhibitor, and vitamin or a vitamin analog;
preferably, the GSK signaling pathway inhibitor is 6-bromoindirubin-3′-oxime;
preferably, the GSK signaling pathway inhibitor is 1-20 μM 6-bromoindirubin-3′-oxime;
preferably, the GSK signaling pathway inhibitor is 10 μM 6-bromoindirubin-3′-oxime;
preferably, the VEGFR kinase inhibitor is selected from the group consisting of SU5402, AV-951, SU5205, and SU5408;
preferably, the VEGFR kinase inhibitor is 1-20 μM SU5402;
preferably, the VEGFR kinase inhibitor is 2 μM SU5402;
preferably, the ROCK pathway is selected from the group consisting of Thiazovivin and Y-27632;
preferably, the ROCK pathway inhibitor is 0.5-20 μM Thiazovivin;
preferably, the ROCK pathway inhibitor is 10 μM Thiazovivin;
preferably, the vitamin or the vitamin analog is selected from the group consisting of biotin, choline chloride, D-calcium pantothenate, folic acid, inositol, nicotinamide, pyridoxine hydrochloride, riboflavin, thiamine hydrochloride, coenzyme Q10, putrescine dihydrochloride, Vitamin A, Vitamin B, Vitamin C, Vitamin D, Vitamin E, Vitamin K, Vitamin H, Vitamin P, Vitamin M, Vitamin T, Vitamin U, and water-soluble vitamins;
preferably, the vitamin or the vitamin analog is Vitamin B;
preferably, the vitamin or the vitamin analog is Vitamin B3;
preferably, the vitamin or the vitamin analog is 1-20 mM Vitamin B3;
preferably, the vitamin or the vitamin analog is 10 mM Vitamin B3;
preferably, a basal culture medium of the RDM3 culture medium is RDM basal culture medium, wherein the RDM basal culture medium comprises DMEM/F12, KSR, Monothioglycerol Solution, Chemically Defined Lipid Concentrate, and glutamine;
preferably, DMEM/F12 is replaced by a cell culture medium selected from the group consisting of William's E medium, Neurobasal Medium, MEM medium, DMEM medium, 1640 RPMI medium, F12 medium, and a mixture thereof;
preferably, KSR is replaced by a serum analog, wherein the serum analog is selected from the group consisting of FBS, horse serum, HAS, and BSA;
preferably, the glutamine is replaced by GlutaMAX™ Supplement or L-glutamine;
preferably, the RDM basal culture medium comprises 88% DMEM/F12, 10% KSR, 5 mM Monothioglycerol Solution, 1% Chemically Defined Lipid Concentrate, and 1% L-glutamine.

2-3. (canceled)

4. A method for quickly and efficiently inducing RPE progenitor cells, comprising culturing stem cells using a culture medium comprising a small molecule compound, wherein the culture medium comprising a small molecule compound is the culture medium according to claim 1;
wherein the small molecule compound is selected from the group consisting of a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, a VEGFR kinase inhibitor, and a mixture thereof;
preferably, the culture medium comprising the small molecule compound is the RDM1 culture medium and/or the RDM2 culture medium;
preferably, the method comprises culturing the cells using the RDM1 culture medium;
preferably, the proportion of cells expressing PAX6 and RPE65 in the cells increases after being cultured in the RDM1 culture medium;
preferably, the proportion of cells expressing PAX6 and RPE65 in the cells increases by 5% after being cultured in the RDM1 culture medium;
preferably, the proportion of cells expressing PAX6 and RPE65 in the cells increases to at least 5% after being cultured in the RDM1 culture medium;
preferably, the method comprises culturing the cells using the RDM2 culture medium;
preferably, the proportion of cells expressing PAX6 and RPE65 in the cells increases after being cultured in the RDM2 culture medium;
preferably, the proportion of cells expressing PAX6 and RPE65 in the cells increases by 5% after being cultured in the RDM2 culture medium;
preferably, the proportion of cells expressing PAX6 and RPE65 in the cells increases from 5% to at least 10% after being cultured in the RDM2 culture medium;
preferably, the expression levels of PAX6, RPE65, IHX2, and pmel17 are increased in the cells cultured in the RDM1 culture medium and/or the RDM2 culture medium.

5. The method according to claim 4, wherein the stem cells are selected from the group consisting of totipotent stem cells, pluripotent stem cells, and unipotent stem cells;
preferably, the stem cells are pluripotent stem cells;
preferably, the stem cells are induced pluripotent stem cells;
preferably, the stem cells are human iPSCs.

6. A method for inducing pigment epithelial precursor cells, comprising culturing cells using a culture medium comprising a small molecule compound, wherein the culture medium comprising a small molecule compound is the culture medium according to claim 1;
wherein the small molecule compound is selected from the group consisting of a GSK signaling pathway inhibitor, a VEGFR kinase inhibitor, a ROCK pathway inhibitor, vitamin or a vitamin analog, and a mixture thereof;
preferably, the culture medium comprising the small molecule compound is the RDM3 culture medium;
preferably, the proportion of cells expressing PAX6 and RPE65 in the cells increases after being cultured in the RDM3 culture medium;
preferably, the proportion of cells expressing PAX6 and RPE65 in the cells increases by 10% after being cultured in the RDM3 culture medium;
preferably, the proportion of cells expressing PAX6 and RPE65 in the cells increases to at least 20% after being cultured in the RDM3 culture medium.

7. The method according to claim 6, wherein the cells cultured using the culture medium comprising the small molecule compound are RPE progenitor cells;
preferably, the RPE progenitor cells are prepared by the method according to claim 4;
preferably, the method further comprises culturing cells using a RDM4 culture medium; wherein the RDM4 culture medium comprises DMEM/F12, KSR, N2 medium, glutamine and vitamin;
preferably, the cells cultured in the RDM4 culture medium is cells that have been cultured in the RDM3 culture medium;
preferably, DMEM/F12 is replaced by a cell culture medium selected from the group consisting of William's E medium, Neurobasal Medium, MEM medium, DMEM medium, 1640 RPMI medium, F12 medium, and a mixture thereof;
preferably, KSR is replaced by a serum analog, wherein the serum analog is selected from the group consisting of FBS, horse serum, HAS, and BSA;
preferably, the glutamine is replaced by GlutaMAX™ Supplement or L-glutamine;
preferably, the RDM4 culture medium comprises 89% DMEM/F12, 10% KSR, 1% N2 medium, 1% L-glutamine and 10 mM Vitamin B3.

8. A method for inducing retinal pigment epithelial cells, comprising at least one of:
1) inducing RPE progenitor cells,
2) inducing pigment epithelial precursor cells,
3) inducing maturation of pigment epithelial cells, and
4) subculturing pigment epithelial cells;
preferably, the step of inducing maturation of retinal pigment epithelial cells comprises culturing pigment epithelial precursor cells using RMM culture medium;
preferably, the step of subculturing retinal pigment epithelial cells comprises culturing mature retinal pigment epithelial cells using REM culture medium.

9. The method according to claim 8, wherein the RMM culture medium comprises DMEM/F12, B27 medium, and glutamine; and the REM culture medium comprises DMEM/F12, KSR, glutamine, β-mercaptoethanol;
preferably, DMEM/F12 is replaced by a cell culture medium selected from the group consisting of William's E medium, Neurobasal Medium, MEM medium, DMEM medium, 1640 RPMI medium, F12 medium, and a mixture thereof;
preferably, KSR is replaced by a serum analog, wherein the serum analog is selected from the group consisting of FBS, horse serum, HAS, and BSA;
preferably, the glutamine is replaced by GlutaMAX™ Supplement or L-glutamine;
preferably, the β-mercaptoethanol is replaced by a reducing agent including but not limited to β-mercaptoethanol, dithiothreitol, dithioerythritol, reduced glutathione, cysteine, thiocarbamate, sodium disulfonate, ascorbate, tin dichloride or sodium borohydride;
preferably, the RMM culture medium comprises 97% DMEM/F12, 2% B27 medium, and 1% L-glutamine;
preferably, the REM culture medium comprises 79% DMEM/F12, 20% KSR, 1% L-glutamine, and 50 μM β-mercaptoethanol.

10. A kit, comprising at least one of the following substances according to claim 1:
1) the BMP signaling pathway inhibitor,
2) the Wnt pathway inhibitor,
3) the inhibitor of TGF-β type I receptors ALK5, ALK4 and ALK7,
4) the ROCK pathway inhibitor,
5) the WNT signaling pathway activator,
6) the VEGFR kinase inhibitor,
7) the GSK signaling pathway inhibitor, and
8) the vitamin or the vitamin.

11. Application, selected from the group consisting of:
1) application of an RDM1 culture medium, an RDM2 culture medium, an RDM3 culture medium in inducing retinal pigment epithelial cells;
2) application of a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, a VEGFR kinase inhibitor, a GSK signaling pathway inhibitor, or vitamin or a vitamin analog in inducing retinal pigment epithelial cells;
3) application of an RDM1 culture medium, an RDM2 culture medium, a BMP signaling pathway inhibitor, a Wnt pathway inhibitor, an inhibitor of TGF-β type I receptors ALK5, ALK4 and ALK7, a ROCK pathway inhibitor, a WNT signaling pathway activator, or a VEGFR kinase inhibitor in inducing RPE progenitor cells; and
4) application of an RDM3 culture medium in inducing pigment epithelial precursor cells.

12. (canceled)

13. Application of the RDM4 culture medium in the method according to claim 5, selected from the group consisting of:
(1) application in inducing retinal pigment epithelial cells; and
(2) application in inducing pigment epithelial precursor cells.

14. Application of the RMM culture medium or the REM culture medium in the method according to claim 6 in inducing retinal pigment epithelial cells.

15. Application of the kit according to claim 8, selected from the group consisting of:
(1) application in inducing retinal pigment epithelial cells;
(2) application in inducing RPE progenitor cells; and
(3) application in inducing pigment epithelial precursor cells.

16. Application of cells prepared by the method according to claim 2 in the manufacture of a medicament for treating an ophthalmic disease;
preferably, the ophthalmic disease includes retinal degenerative disease;
preferably, the retinal degenerative disease includes retinitis pigmentosa, macular degeneration, Leber disease, Usher syndrome, and retinal atrophy;
preferably, the macular degeneration includes juvenile macular degeneration and age-related macular degeneration.

17. Application of cells prepared by the method according to claim 3 in the manufacture of a medicament for treating an ophthalmic disease;
preferably, the ophthalmic disease includes retinal degenerative disease;
preferably, the retinal degenerative disease includes retinitis pigmentosa, macular degeneration, Leber disease, Usher syndrome, and retinal atrophy;
preferably, the macular degeneration includes juvenile macular degeneration and age-related macular degeneration.

18. Application of cells prepared by the method according to claim 4 in the manufacture of a medicament for treating an ophthalmic disease;
preferably, the ophthalmic disease includes retinal degenerative disease;
preferably, the retinal degenerative disease includes retinitis pigmentosa, macular degeneration, Leber disease, Usher syndrome, and retinal atrophy;
preferably, the macular degeneration includes juvenile macular degeneration and age-related macular degeneration.

19. Application of cells prepared by the method according to claim 5 in the manufacture of a medicament for treating an ophthalmic disease;
preferably, the ophthalmic disease includes retinal degenerative disease;
preferably, the retinal degenerative disease includes retinitis pigmentosa, macular degeneration, Leber disease, Usher syndrome, and retinal atrophy;
preferably, the macular degeneration includes juvenile macular degeneration and age-related macular degeneration.

20. Application of cells prepared by the method according to claim 6 in the manufacture of a medicament for treating an ophthalmic disease;
preferably, the ophthalmic disease includes retinal degenerative disease;
preferably, the retinal degenerative disease includes retinitis pigmentosa, macular degeneration, Leber disease, Usher syndrome, and retinal atrophy;
preferably, the macular degeneration includes juvenile macular degeneration and age-related macular degeneration.

21. Application of cells prepared by the method according to claim 7 in the manufacture of a medicament for treating an ophthalmic disease;
preferably, the ophthalmic disease includes retinal degenerative disease;
preferably, the retinal degenerative disease includes retinitis pigmentosa, macular degeneration, Leber disease, Usher syndrome, and retinal atrophy;
preferably, the macular degeneration includes juvenile macular degeneration and age-related macular degeneration.

22. A cell population prepared by the method according to claim 4, wherein the proportion of cells expressing PAX6 and RPE65 in the cell population is at least 5%, preferably at least 10%.

23. A cell population prepared by the method according to claim 6, wherein the proportion of cells expressing PAX6 and RPE65 in the cell population is at least 10%, preferably at least 20%.

24. A cell population prepared by the method according to claim 8, selected from the group consisting of
1) a cell population, wherein the proportion of positive cells expressing ZO-1, RPE65, Pax6, and CRALBP in the cell population is at least 80%; and
2) a cell population, wherein the expression levels of TYRP2, PEDF, PMEL17, RPE65, and CRALBP in the cell population are increased by at least 100 times relative to iPSCs.
